(19) United States
(12) Patent Application Publication
(10) Pub. No.: US 2023/0011880 A1
(43) Pub. Date: Jan. 12, 2023

(54) SYSTEM AND METHOD FOR REAL-TIME PREDICTION OF HOSPITAL DISCHARGE DISPOSITION AND DEFERRING CLINICAL SERVICES

(71) Applicant: KONINKLIJKE PHILIPS N.V., EINDHOVEN (NL)

(72) Inventors: Lasith Adhikari, Cambridge, MA (US); David Paul Noren, Cambridge, MA (US); Gregory Boverman, Cambridge, MA (US); Eran Simhon, Cambridge, MA (US); Chaitanya Kulkarni, Bangalore (IN); Syamanthaka Balakrishnan, Bangalore (IN); Vikram Shivanna, Bangalore (IN); Larry James Eshelman, Briarcliff Manor, NY (US); Kailash Swaminathan, Bengaluru (IN)

(21) Appl. No.: 17/856,058

(22) Filed: Jul. 1, 2022

Related U.S. Application Data
(60) Provisional application No. 63/218,523, filed on Jul. 6, 2021.

(51) Int. Cl. G16H 10/60; G16H 50/20; G16H 50/70

(52) U.S. Cl. CPC G16H 10/60; G16H 50/20; G16H 50/70; G16H 40/20

(57) ABSTRACT

A method for performing, using a patient disposition system, a disposition analysis of a plurality of patients to optimize a discharge planning process for each of the plurality of patients, including: (i) receiving electronic medical record information about each of the plurality of patients; (ii) identifying one of a plurality of different patient types for each of the plurality of patients based on the received electronic medical record information; (iii) selecting a trained multi-state model for each identified patient type; and (iv) determining, based on the selected trained multi-state model, a disposition state for each of the plurality of patients in real-time, wherein each disposition state includes a location to which the patient is to be discharged. The method further includes determining at least one service or assessment that can be deferred to the location to which the patient is to be discharged.

### FIELD OF THE DISCLOSURE

The present disclosure is directed generally to methods and systems for optimizing the discharge planning process for patients in a hospital.

### BACKGROUND

Hospital stays are very expensive for patients and healthcare costs are expected to continue to grow annually. To meet the increasing demand and reduce healthcare costs, it is vital to optimize the discharge planning process and reduce unnecessary hospital days. Efforts to initiate early discharge planning and reduce the length of stay (LOS) require accurate preplanning of patient discharge location during the early stages of the stay. Prolonged and unnecessary hospital stays can result from ineffective or delayed discharge planning, including the identification of a patient's discharge disposition.

Recently, the “flip your discharge” concept has received significant attention, highlighting the cost and clinical effectiveness of deferring hospital services or assessments to home health care whenever possible. The idea behind this concept is to defer in-hospital discharge assessments/services to home services with no increase in readmission or any other poor healthcare outcomes. Although hospitals are presently developing and deploying different tools to determine the anticipated discharge location for different patient profiles, these tools do not provide insight into the earliest time that a patient can be discharged to that location.

Unfortunately, identifying which services can be deferred or carried out outside the hospital is challenging and must occur early in the patient stay to avoid discharge delay. Identifying which services or assessments can be deferred is intimately linked to the discharge location level of care. Moreover, the availability of specific services is dependent on the patient's discharge location (i.e., home, skilled nursing facility, rehab, etc.).

### SUMMARY OF THE DISCLOSURE

There is a continued need for methods and systems that predict a most likely discharge disposition for a plurality of hospital patients in real-time regardless of patient type, the discharge disposition including a discharge location. There is also a need for methods and systems that then determine if any services or assessments needed can be deferred to when the patient is at the predicted discharge location.

The present disclosure is directed at inventive methods and systems for initiating early discharge planning and reducing a patient's length of stay in a hospital. Various embodiments and implementations herein are directed to a system or method that comprises modeling to predict a patient's discharge disposition in real-time based on a type of injury the patient has sustained, and recommending one or more locations, one or more providers, and/or one or more services to be utilized after the patient is discharged based on available post-hospital service provider information and sociodemographic information and economic factors for the patient. The system or method receives information relevant to a patient, including the type of injury the patient has sustained and other clinical observations. The system or method matches the information relevant to the patient with available trained multi-state models and generates a predicted discharge disposition, including a location to which a patient is to be discharged and optionally any services or assessments which can be deferred to the location to which the patient is to be discharged. The system or method transmits real-time alerts to the patient's family, the alerts including the most up-to-date discharge disposition and post hospital service requirements, if any. The system or method also simultaneously transmits the real-time predicted disposition state information to a display accessible by the patient's care manager or care team and the post hospital service requirements, if any.

Generally, in one aspect, a method for performing, using a patient disposition system, a disposition analysis of a plurality of patients to optimize a discharge planning process for each of the plurality of patients is provided. The method includes receiving, by the patient disposition system, electronic medical record information about each of the plurality of patients, the electronic medical record information comprising at least one clinical observation about each of the plurality of patients; identifying, by a processor of the patient disposition system, one of a plurality of different patient types for each of the plurality of patients based on the received electronic medical record information; selecting, by the processor of the patient disposition system, a trained multi-state model for each identified patient type; determining, by the processor of the patient disposition system based on the selected trained multi-state model, a disposition state for each of the plurality of patients in real-time, wherein each disposition state comprises a location to which the patient is to be discharged; and transmitting the disposition state for each of the plurality of patients in real-time to at least one caregiver of each of the plurality of patients, wherein the disposition state is configured to assist the at least one caregiver in discharging each of the plurality of patients.

According to an embodiment, a first patient of the plurality of patients is associated with at least one clinical observation that is indicative of a first patient type and the plurality of patients further comprises at least a second patient that is associated with at least one other clinical observation that is indicative of a second patient type, wherein the first and second patient types have different clinical profiles.

According to an embodiment, the method further includes determining, by the processor of the patient disposition system, at least one service or assessment that can be deferred to the location to which at least one patient is to be discharged.

According to an embodiment, the method further includes training at least one multi-state model for a first patient type, the training comprising: (i) identifying a first patient type based on electronic medical record information comprising patient information; (ii) extracting relevant data elements from the electronic medical record information; (iii) preprocessing the extracted relevant data elements to generate a clean dataset; (iv) splitting the clean dataset into a training data cohort and a test data cohort; and (v) training the at least one multi-state model using the training data cohort.

According to an embodiment, the splitting the clean dataset into the training data cohort and the test data cohort comprises randomly splitting the clean dataset such that a first amount of the clean dataset in the training data cohort is different than a second amount of the clean dataset in the test data cohort.

According to an embodiment, the training data cohort comprises a set of observation sequences and training the at least one multi-state model comprises determining at least one optimal parameter for maximizing a probability of observing the set of observation sequences.

According to an embodiment, the method further includes generating, by the patient disposition system, a patient disposition report comprising at least one determined disposition state for at least one patient of the plurality of patients in real-time.

According to an embodiment, the method further includes displaying the patient disposition report on a display of the patient disposition system in real-time or transmitting a notification regarding the at least one disposition state to a family member of the patient in real-time.

According to an embodiment, the method further includes receiving, updated electronic medical record information for at least one patient of the plurality of patients; updating, by the trained multi-state model, the disposition state for the at least one patient based on the received updated electronic medical record information; and discharging the at least one patient to the location.

According to an embodiment, the method further includes receiving or accessing, by the patient disposition system, sociodemographic data about the patient; receiving or accessing, by the patient disposition system, information about post-hospital service providers; determining, by the processor of the patient disposition system, at least one relevant or convenient service or assessment to be provided based on the information about the post-hospital service providers and the sociodemographic data about the patient; and obtaining medical services from at least one post-hospital service provider for the patient such that treatment is provided to the patient.

According to another aspect, a patient disposition system configured to perform a disposition analysis of a plurality of patients to optimize a discharge planning process for each of the plurality of patients is provided. The system includes a plurality of multi-state models, each trained to generate a disposition state for a patient; electronic medical record information about the plurality of patients, the electronic medical record information comprising at least one clinical observation about each of the plurality of patients; and a processor configured to: (i) identify a patient type for each patient of the plurality of patients based on the electronic medical record information; (ii) select a trained multi-state model for each identified patient type; (iii) determine, based on the selected trained multi-state model, a disposition state for each patient of the plurality of patients in real-time, wherein each disposition state comprises a location to which the patient is to be discharged; and (iv) transmit the disposition state for each of the plurality of patients in real-time to at least one caregiver of each of the plurality of patients, wherein the disposition state is configured to assist the at least one caregiver in discharging each of the plurality of patients.

According to an embodiment, the plurality of patients comprises a first patient that is associated with at least one clinical observation that is indicative of a first patient type and the plurality of patients comprises at least a second patient that is associated with at least one other clinical observation that is indicative of a second patient type, wherein the first and second patient types have different clinical profiles.

According to an embodiment, the processor of the system is further configured to determine at least one service or assessment that can be deferred to the location to which at least one patient is to be discharged.

According to an embodiment, the processor of the system is further configured to generate a patient disposition report comprising at least one determined disposition state for at least one patient of the plurality of patients in real-time.

According to an embodiment, the system further includes a display configured to display the patient disposition report.

In various implementations, a processor or controller may be associated with one or more storage media (generically referred to herein as “memory,” e.g., volatile, and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for performing a patient disposition analysis for a plurality of patients using a patient disposition system, in accordance with an embodiment.

FIG. 2 is a flowchart of a method for training and validating a multi-state model for a given patient type, in accordance with an embodiment.

FIG. 3 is a flowchart of a method for predicting a discharge state for a new patient, in accordance with an embodiment.

FIG. 4 is a flowchart of a method for providing user interaction with the system, in accordance with an embodiment.

FIG. 5 is a schematic representation of a patient disposition system, in accordance with an embodiment.

FIG. 6 is a flowchart of a method for performing a patient disposition analysis for a plurality of patients using a patient disposition system, in accordance with an embodiment.

FIG. 7 is a schematic representation of a patient disposition system, in accordance with an embodiment.

FIG. 8 is a schematic representation of a patient disposition system, in accordance with an embodiment.

### DETAILED DESCRIPTION OF EMBODIMENTS

The present disclosure describes various embodiments of a system and method for generating a patient's discharge disposition report comprising a location to which the patient is to be discharged and one or more services or assessments that can be provided at the location to which the patient is to be discharged, if any. Applicant has recognized and appreciated that it would be beneficial to provide a method and system that can predict a discharge disposition state for a patient early in the patient's stay to reduce the patient's length of stay. Applicant has further recognized and appreciated that it would be beneficial to provide a method and system that can help a clinical care team to predict the most probable discharge disposition for any given type of patient at any time during their stay in the hospital and which also identifies if any care services can be deferred. The system or method receives clinical information relevant to a patient, including the type of injury or procedure the patient has experienced. The system or method matches the clinical information with available trained multi-state models and generates a predicted discharge disposition. The predicted discharge disposition includes a location to which a patient is to be discharged and any services or assessments which can be deferred to the location to which the patient is to be discharged. The system or method generates and transmits real-time alerts to the patient's family and updates to the patient's care manager or care team.

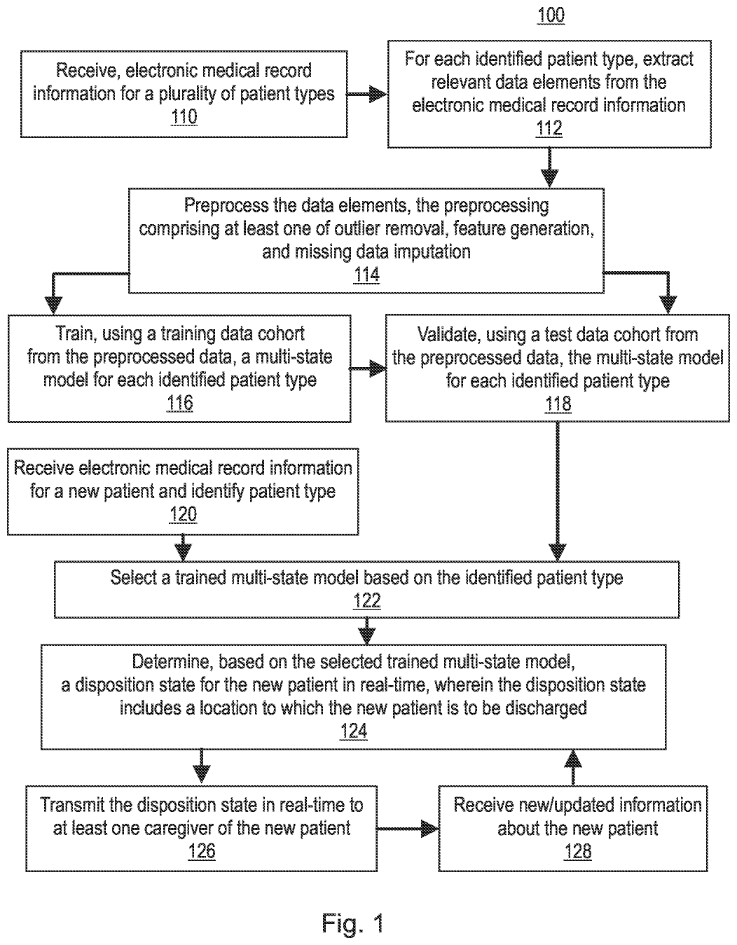

Referring to FIG. 1, in one embodiment, is a flowchart of a method 100 for performing, using a patient disposition system, a patient disposition analysis of a plurality of patients to optimize a discharge planning process for each of the plurality of patients. The patient disposition system may be any patient disposition system described or otherwise envisioned herein.

As discussed in greater detail herein, the patient disposition system comprises multi-state models that have been trained to determine or predict a disposition state of a patient, the disposition state including a location to which the patient is to be discharged and optionally any services or assessments that can be deferred to the location. In embodiments, given a known model, which depends on a patient type, and a sequence of observations (e.g., observed clinical events, demographics, procedures, etc.), an optimal hidden state sequence can be generated in real-time. In other words, the patient disposition system is configured to use electronic medical record information including at least one clinical observation and trained multi-state models to determine where the patient is going to be discharged in real-time. The clinical observations can include any medical information, such as, procedures, status post-procedures or surgeries, complications, etc. The disposition state can be utilized by the people providing care to the patient in the hospital and/or the patient's family members to facilitate discharge planning. The multi-state models of the patient disposition system can be trained using medical record information for a variety of patient types (e.g., after critical care, post-acute palliative care, after stroke, after hip fracture surgery, after total hip and/or knee arthroplasty, after chest pain, after brain injury, after fall), the medical record information including, for each of the patient types, medical information about the patients, an indication of a location to which they are discharged, the time of the discharge event, and information about the care received at the location to which they are discharged. Features are extracted from the medical record information and utilized to train the multi-state models of the patient disposition system.

According to an embodiment, the patient disposition system may be embodied in whole or in part within a device. For example, the entire patient disposition system may be embodied within a single device such as a handheld device, laptop, computer, or other single device. Alternatively, the patient disposition system may comprise a user interface that is transportable, such as a handheld device, mobile phone application, computer, or other transportable element that functions as a user interface to receive information proximate to the patient. The device will communicate the information to another, remote component of the patient disposition system for analysis. The results of the patient disposition system may then be communicated back to the transportable user interface.

At step 110 of the method, the patient disposition system receives input data comprising training data about the discharge disposition of a plurality of patient types. The training data can comprise medical record information about each of the patient types, including but not limited to demographics, physiological measurements such as vital data, injury information, physical observations, and/or diagnosis, among many other types of medical information. The medical record information may include information obtained at the location where the patient received care as well as medical record information obtained after discharge. As an example, the medical record information can include detailed information on patient demographics such as age, gender, socioeconomic factors, payment source, length of stay, disposition, comorbidities, orders; for brain injury patients: Glasgow Coma Scale, injury severity score, and more; for hip fracture surgery patients: type of fracture, type of surgery etc.; clinical notes (e.g., prescribed services/assessments at or near discharge); diagnosis or medical condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types of medical information are possible.

This training data may be stored in and/or received from one or more databases. The database may be a local and/or remote database. For example, the patient disposition system may comprise a database of training data.

According to an embodiment, the patient disposition system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 112 of the method, the system identifies a patient type for the medical record information pertaining to each patient. A patient type refers to the condition of the patient prior to discharge (e.g., after stroke, after hip fracture surgery, chest pain, brain injury, after fall, etc.). The system also extracts relevant data elements from the electronic medical record information for each identified patient type. This can be accomplished by a variety of embodiments for feature identification, extraction, and/or processing, including any method for extracting features from a dataset. The outcome of a feature processing step or module of the patient disposition system is a set of patient features related to medical record information about a patient and discharge information and/or outcome, which thus comprises a dataset that can be utilized to train a multi-state model for the patient type.

At step 114 of the method, the system performs data preprocessing to generate a clean dataset. The preprocessing includes at least one of outlier removal, feature generation, and missing data imputation, etc. to generate a dataset that represents the ideal data elements corresponding to the patient type and based on the extracted relevant data elements. The preprocessing can be implemented by any suitable methods for multi-state models.

At step 116 of the method, the system trains each of the patient type multi-state models, which will be the models utilized to analyze new patient electronic medical record information as described or otherwise envisioned herein. Each multi-state model is trained using a portion of the generated clean dataset. In embodiments, the generated clean dataset is randomly split into a training data cohort and a test data cohort and the training data cohort is used to train the multi-state model. In embodiments, the generated clean dataset is randomly split such that a first amount of the clean dataset in the training data cohort is different than a second amount of the clean dataset in a test data cohort. The portion of the clean dataset in the test data cohort will be used to validate each of the multi-state models. For example, 60-80% of the clean dataset can be apportioned for the training data cohort and 20-40% of the clean dataset can be apportioned for the test data cohort. In embodiments, 70% of the clean dataset can be apportioned for the training data cohort and 30% of the clean dataset can be apportioned for the test data cohort.

Since a patient's most preferable state related to the disposition is not known at any given time and cannot be found in the data, embodiments utilize Hidden Markov Models (HMM) to model randomly changing systems, where the system states are at least partially not observable. Hidden Markov Models are stochastic models. Here, for example, we can observe a sequence of clinical events from a patient, but the clinical events could be linked to: (i) a state of discharge to home with oxygen or (ii) a state of discharge to home with nursing care. For the same sequence of observations, there may be many possible patient discharge state sequences that could have been generated. The HMM is configured to track the observed features in the clinical observations and their interactions over time and outputs a sequence of states which has the highest probability of generating the observations.

In embodiments, an HMM has the following parameters: a number of states in the model, initial state probabilities, state transition probabilities, and observation probabilities as further explained below.

The number of states in the model can be represented as S={S₁, S₂, . . . , S_{N}}, where S can be, for example, {home; home+oxygen (O₂); home+nursing care; rehab; rehab+X-ray; rehab+physical therapy, long-term care (LTC); LTC+physical therapy, . . . } and n is a number equal to or greater than 1. A state can represent a location alone, for example, home, rehab, or long-term care facility. A state can also represent a location with medical services, for example, home and oxygen or home and nursing care.

The initial state probabilities can be represented as follows: Π=[πᵢ]w, where πᵢ≡Pr(State at time₀=Sᵢ).

The state transition probabilities can be represented as follows: A=[aᵢⱼ] where aᵢⱼ≡Pr(State at timeₜ₊₁=Sⱼ|State at timeₜ=Sᵢ).

The observation probabilities can be represented as follows: B=[bⱼ(m)] where bⱼ(m)≡Pr(Observation m at timeₜ|State at timeₜ=Sⱼ).

The full HMM can be written as λ=(A, B, Π), where the model represents the state transition probabilities, the observation probabilities, and the initial state probabilities. With a given training set of observation sequences χ, the model λ is trained (i.e., to find optimal parameters (A, B, Π)) such that those parameters maximize the probability of observing the given training set of observation sequences χ. Any suitable algorithm can be used to solve for the unknown optimal parameters (A, B, Π), for example, the Baum-Welch algorithm.

At step 118 of the method, the system validates the multi-state model for each identified patient type using the test data cohort described above from the clean dataset generated.

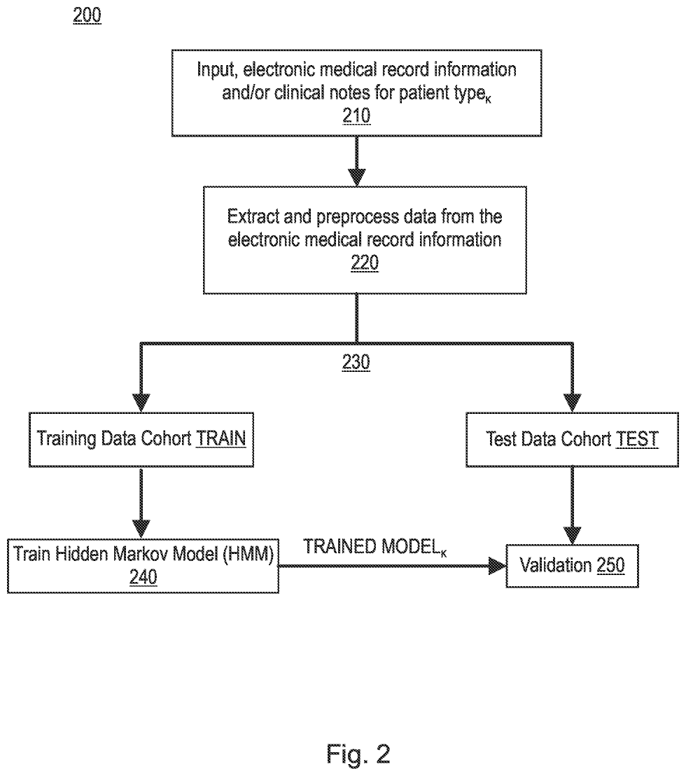

FIG. 2 shows an example process 200 of training and validating a Hidden Markov Model (HMM) for a given patient type PATIENT TYPE_{K} using a patient disposition system as described herein. At step 210 of the process, the system receives, retrieves, obtains, or otherwise accesses electronic medical record information and/or clinical notes for a patient and uses the information and/or clinical notes as input data. At step 220 of the process, the system extracts relevant data elements from the information and/or clinical notes and performs the preprocessing techniques to generate a clean dataset. At step 230, the system randomly splits the clean dataset into a training data cohort TRAIN and a test data cohort TEST as described herein. At step 240 of the process, the system trains the HMM_{K} as described herein with the training data cohort TRAIN. At step 250 of the process, the system performs a validation of TRAINED MODEL_{K} using the test data cohort TEST.

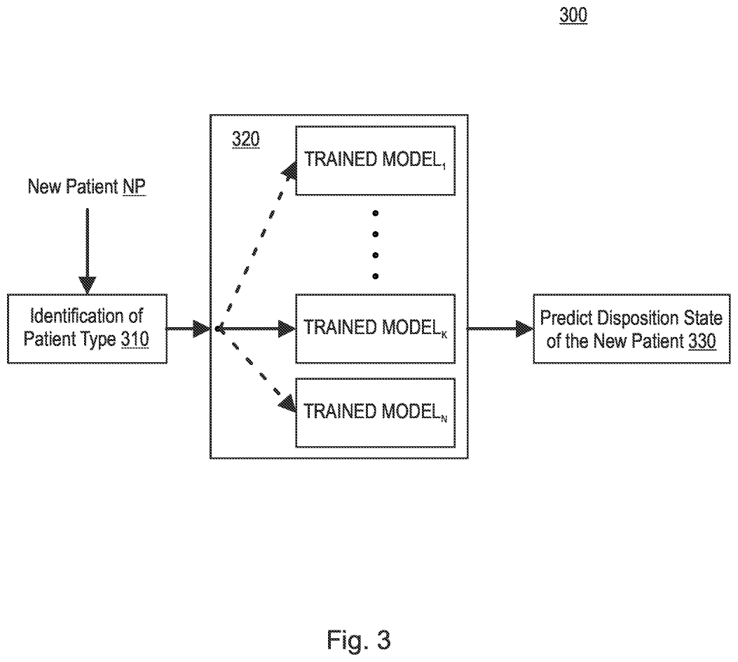

After the models are trained for the different patient types, the patient disposition system includes different trained models that can be utilized to predict a disposition state of a new patient as shown in FIGS. 1 and 3 in real-time. The trained multi-state models can be static such that they are trained once and are utilized for the real-time predictions. According to another embodiment, the trained multi-state models can be more dynamic such that they are updated or re-trained using subsequently available training data. The updating or re-training can be constant or can be periodic.

As shown in FIG. 3, at step 310, when a new patient NP is admitted to a hospital or ward, a patient type is identified using an admitting diagnosis, working diagnosis, or a patient's problem list. Any suitable information can be used to identify a patient type. As part of the patient type identification, a new input patient profile can be created. In FIG. 1, at step 120, the system receives electronic medical record information for a new patient and identifies patient type (e.g., after stroke, after hip fracture surgery, chest pain, brain injury, after fall).

At step 122, the system selects a trained multi-state model based on the identified patient type. At step 320, the system performs a patient-to model matching to select a trained multi-state model. The new input patient profile can be matched with the available trained models. In other words, based on the identified patient type, the patient information will be matched with the pre-trained HMI models deployed in the system. In embodiments, if there is not match between the identified patient type and the pre-trained HMM models, the system can use a default model that is based on a variety of different patient types. In embodiments, the system can read all the required data elements based on the selected model on-the-fly and assists many different patient types in predicting discharge disposition states more accurately.

At step 330, the system generates the optimal hidden state sequence in real-time based on the known selected model and sequence of observations (e.g., observed clinical events, demographics, procedures, etc.). In embodiments, the system provides a plug-and-play environment for the pre-trained multi-state prediction models. The system generates a predicted disposition state for the new patient NP in real-time, the predicted disposition state including a discharge location (i.e., home, rehabilitation care, or long-term facility) along with potential services/assessments that can be deferred to the discharge location. In FIG. 1, at step 124, the system determines a discharge disposition state for the new patient in real-time based on the selected trained multi-state model. The discharge disposition state includes a location to which the new patient is to be discharged and any assessments or services that can be provided at the location as explained further herein. In embodiments, the services or assessments can be based on the location to which the patient is to be discharged. In embodiments, the disposition state can be based at least in part on a cost analysis.

At step 126, the system transmits the disposition state in real-time to at least one caregiver of the new patient. The disposition state may comprise many different configurations and information. For example, the disposition state comprises the determined location to which the patient is expected to be discharged and optionally any hospital services or assessments that can be deferred to the determined location. The system can also be configured to display the disposition state on a display of the system. The display may comprise information about one or more locations to which the patient is expected to be discharged, and expected future hospital assessments or services that might be able to be deferred to the one or more locations. The display may further comprise an indication of which of two or more locations to discharge the patient, and/or an estimated time for the patient to reach the recommendation location. Other information is possible as well. Alternatively, the disposition state may be communicated by wired and/or wireless communication to another device. For example, the system may communicate the disposition state to a mobile phone, computer, laptop, wearable device, and/or any other device configured to allow display and/or other communication of the disposition state.

According to a possible embodiment, at step 128 of the method, the system may receive new or updated information about the new patient. The updated or new information may be the same type of electronic medical record information received at step 120. For example, the status of a patient may change for the better or the worse which indicates that a new patient disposition state may be necessary. That updated medical information, such as worsening vital signs or improved prognosis, may be provided to the system. As another example, the status of a post-hospital service provider may change, due to an event such as capacity being filled, and that information may be provided to the system.

With the updated information, the system returns to step 124 of the method to update the discharge disposition state for the new patient in real-time based on the new or updated information and the selected trained multi-state model. If the discharge disposition state has changed, the system transmits the new disposition state in real-time to the at least one caregiver of the patient.

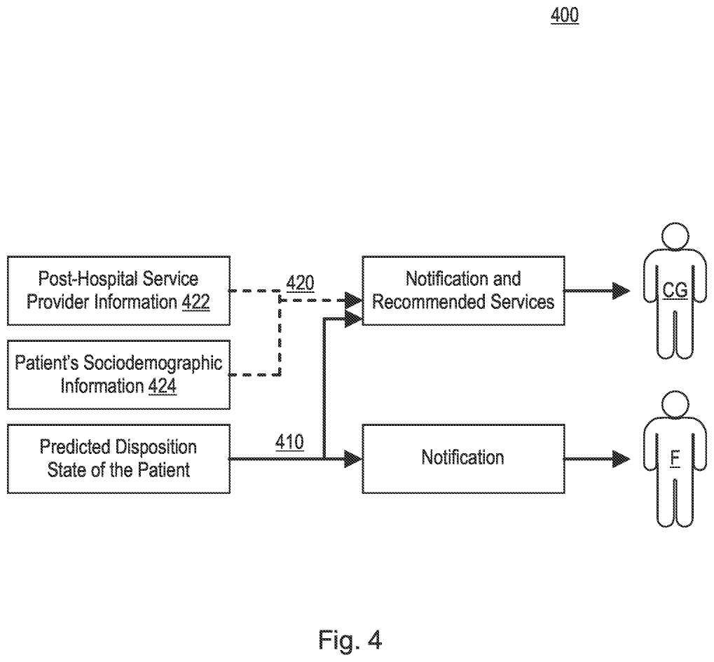

FIG. 4 shows a flowchart for providing user interaction with the patient disposition systems described herein. At step 410, the predicted disposition state of the patient is sent in real-time to the family members F of the patient. The family members F can be notified through phone or email alerts or any other suitable alternative. Simultaneously, the predicted disposition state of the patient is sent in real-time to the caregiver CG of the patient (e.g., a care manager/care team). The caregiver CG can receive the predicted disposition state on a dashboard display. Optionally, at step 420, if the system has access to information regarding post-hospital service providers 422 and the patient's sociodemographic data 424, the system will determine and recommend the most convenient and relevant services based on the patient's sociodemographic data.

According to embodiments, the patient disposition system receives capability information for post-hospital service providers in a predetermined vicinity of the hospital. The capability information, for example, can include information about the provider's capacity and a capability to treat the patient. Capacity information can be information about the ability of the provider to accept new patients. Capability information can be information about the ability of the provider to handle or treat the patients. For example, some providers may not be equipped at a certain time to handle certain types of injuries. Capacity and/or capability information may be pre-programmed into the patient disposition system or it may be obtained periodically or continually by communication with a database or other source of this information. For example, providers may maintain a database of current capacity and capabilities, and the patient disposition system may be configured to query that database to obtain that information.

The predetermined vicinity may be any range that is suitable depending on the area and the type of injury and services desired. This may be pre-programmed, such as by a user or other entity defining an allowable distance, or may be based on information gleaned due to threshold parameters of the patient disposition system.

The received information may be stored in one or more databases, which may be local and/or remote databases. For example, the patent disposition system may comprise a database or other memory configured to store the data.

Thus, the system optimizes the patient discharge disposition of each patient regardless of patient type and at any time during the patient's stay in the hospital. As described herein, the system predicts the disposition state of inpatients with different clinical profiles in real-time. The disposition state comprises the hospital discharge location and potential services to be deferred (e.g., doctor care at home for diagnosing and treating one or more illnesses, nursing care at home: intravenous therapy, drug administration, pain control, wound dressing, etc., physical, occupational, and/or speech therapy at home or at a rehabilitation facility, laboratory (blood test, urine test, etc.) and portable X-ray imaging at home or at a long-term care facility). Given the predicted disposition state, a patient's clinical care team will be able to identify the most suitable post hospital care and/or services based on the patient's demographics and socioeconomic factors. Additionally, the patient's family will be able to know the anticipated discharge location and potential post hospital services required to be setup at home (e.g., home oxygen/ventilator services) in advance.

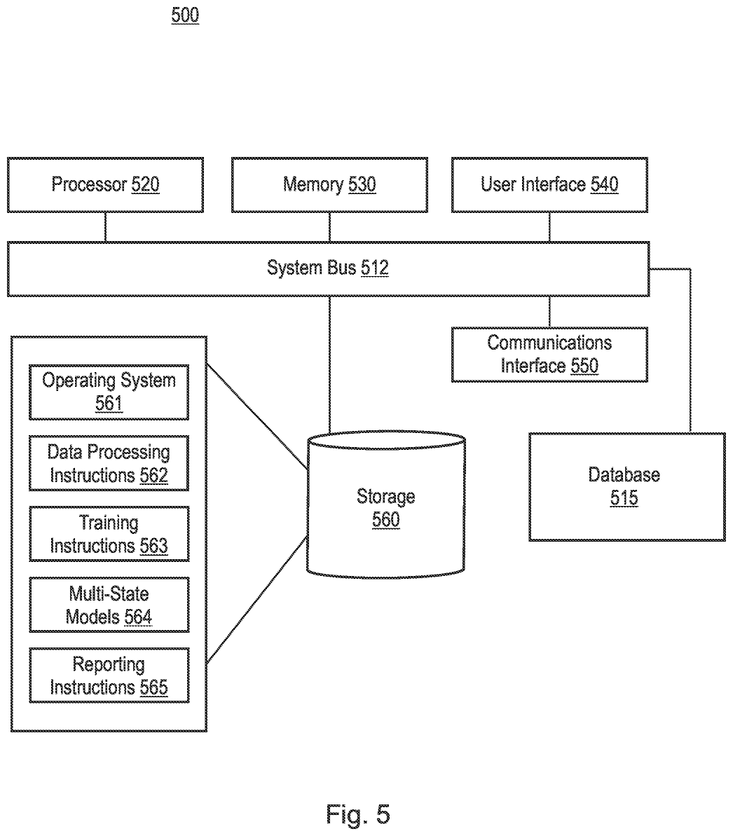

Referring to FIG. 5, in one embodiment, is a schematic representation of a patient disposition system 500. System 500 may be any of the systems described or otherwise envisioned herein, and may comprise any of the components described or otherwise envisioned herein.

According to an embodiment, system 500 comprises one or more of a processor 520, memory 530, user interface 540, communications interface 550, and storage 560, interconnected via one or more system buses 512. It will be understood that FIG. 5 constitutes, in some respects, an abstraction and that the actual organization of the components of the system 500 may be different and more complex than illustrated.

According to an embodiment, system 500 comprises a processor 520 capable of executing instructions stored in memory 530 or storage 560 or otherwise processing data to, for example, perform one or more steps of the method. Processor 520 may be formed of one or multiple modules. Processor 520 may take any suitable form, including but not limited to a microprocessor, microcontroller, multiple microcontrollers, circuitry, field programmable gate array (FPGA), application-specific integrated circuit (ASIC), a single processor, or plural processors.

Memory 530 can take any suitable form, including a non-volatile memory and/or RAM. The memory 530 may include various memories such as, for example L1, L2, or L3 cache or system memory. As such, the memory 530 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 500. It will be apparent that, in embodiments where the processor implements one or more of the functions described herein in hardware, the software described as corresponding to such functionality in other embodiments may be omitted.

User interface 540 may include one or more devices for enabling communication with a user. The user interface can be any device or system that allows information to be conveyed and/or received, and may include a display, a mouse, and/or a keyboard for receiving user commands. In some embodiments, user interface 540 may include a command line interface or graphical user interface that may be presented to a remote terminal via communication interface 550. The user interface may be located with one or more other components of the system, or may located remote from the system and in communication via a wired and/or wireless communications network.

Communication interface 550 may include one or more devices for enabling communication with other hardware devices. For example, communication interface 550 may include a network interface card (NIC) configured to communicate according to the Ethernet protocol. Additionally, communication interface 550 may implement a TCP/IP stack for communication according to the TCP/IP protocols. Various alternative or additional hardware or configurations for communication interface 550 will be apparent.

Storage 560 may include one or more machine-readable storage media such as read-only memory (ROM), random-access memory (RAM), magnetic disk storage media, optical storage media, flash-memory devices, or similar storage media. In various embodiments, storage 560 may store instructions for execution by processor 520 or data upon which processor 520 may operate. For example, storage 560 may store an operating system 561 for controlling various operations of system 500.

It will be apparent that various information described as stored in storage 560 may be additionally or alternatively stored in memory 530. In this respect, memory 530 may also be considered to constitute a storage device and storage 560 may be considered a memory. Various other arrangements will be apparent. Further, memory 530 and storage 560 may both be considered to be non-transitory machine-readable media. As used herein, the term non-transitory will be understood to exclude transitory signals but to include all forms of storage, including both volatile and non-volatile memories.

While system 500 is shown as including one of each described component, the various components may be duplicated in various embodiments. For example, processor 520 may include multiple microprocessors that are configured to independently execute the methods described herein or are configured to perform steps or subroutines of the methods described herein such that the multiple processors cooperate to achieve the functionality described herein. Further, where one or more components of system 500 is implemented in a cloud computing system, the various hardware components may belong to separate physical systems. For example, processor 520 may include a first processor in a first server and a second processor in a second server. Many other variations and configurations are possible.

According to an embodiment, system 500 may comprise or be in remote or local communication with a database or data source 515. Database 515 may be a single database or data source or multiple databases or data sources. Database 515 may comprise the input data which may be used to train the multi-state models, as described and/or envisioned herein. The input data may also be the data for a specific patient for which the disposition state analysis is being performed. As an example, the input data can include detailed information on patient demographics such as age, gender, and more; diagnosis or medical condition such as cardiac disease, psychological disorders, chronic obstructive pulmonary disease, and more; physiological vital signs such as heart rate, blood pressure, respiratory rate, oxygen saturation, and more; and/or current and/or past data. Many other types of input data are possible. Accordingly, database 515 may be an electronic medical record database or any other type of database.

According to an embodiment, storage 560 of system 500 may store one or more algorithms, models, and/or instructions to carry out one or more functions or steps of the methods described or otherwise envisioned herein. For example, processor 520 may comprise one or more of data processing instructions 562, training instructions 563, multi-state models 564, and/or reporting instructions 565.

According to an embodiment, data processing instructions 562 direct the system to retrieve and process input data which is used to either: (i) train the multi-state models 564 using the training instructions 563, or (ii) to perform a discharge disposition analysis for the patient using the trained multi-state models 564. The data processing instructions 562 direct the system to, for example, receive or retrieve input data or medical data to be used by the system as needed, such as from database 515 among many other possible sources. As described above, the input data can comprise a wide variety of input types from a wide variety of sources.

According to an embodiment, the data processing instructions 562 also direct the system to process the input data to generate a plurality of features related to medical information for a plurality of patients, which are used to train the multi-state models 564. This can be accomplished by a variety of embodiments for feature identification, extraction, and/or processing. The outcome of the feature processing is a set of features related to discharge information and/or outcome for a cohort of previously monitored patients, which thus comprises a training data set that can be utilized to train the multi-state models.

According to an embodiment, training instructions 563 direct the system to utilize the processed data to train the multi-state models to determine a patient disposition state for a patient, wherein the patient disposition state for a patient comprises a location to which the patient is to be discharged in real-time. The multi-state models can be any suitable model sufficient to utilize the type of input data provided. Thus, the system comprises trained multi-state models 564 configured to determine a discharge disposition state for a patient.

Each of the trained multi-state models 564 is configured to determine a discharge disposition state for a patient of a patient type using the received electronic medical record information. The disposition state can comprise, for example, at least one or two or more locations to which the patient can be discharged and a recommendation of which locations the patient should be discharged to. Preferably, the patient discharge disposition system and the trained multi-state models 564 make disposition states for patients that defer the largest number of in-hospital discharge assessments/services with no increase in readmissions or any other adverse healthcare outcomes.

According to an embodiment, reporting instructions 565 direct the system to generate and provide a disposition state report. The disposition state report comprises the determined disposition state for each patient. The disposition state report may comprise many different configurations and information. For example, the disposition state report may comprise the one or more locations to which the patient can be discharged, and a recommendation to discharge the patient to one of the one or more locations. The disposition state report may further comprise an estimated time for the patient to be discharged to the recommended location or any of the one or more locations. The disposition state report may also comprise a recommendation to defer one or more in-hospital assessments or services to the one or more discharge locations. The reporting instructions 565 also direct the system to display the disposition state report on a display of the system. The display may comprise information about the locations to which the patient is to be discharged, the time to discharge, and the availability of in-hospital assessments and services at the locations. The display may further comprise an indication of which of the locations to discharge the patient, and/or which of the in-hospital assessments or services should be deferred. Other information is possible. Alternatively, the disposition state report may be communicated by wired and/or wireless communication to another device. For example, the system may communicate the disposition state report to a mobile phone, computer, laptop, wearable device, and/or any other device configured to allow display and/or other communication of the disposition state report.

According to an embodiment, the patient discharge disposition state system is configured to process many thousands or millions of datapoints in the input data used to train the multi-state models, as well as in the received electronic medical record information utilized for disposition states for a plurality of patients. For example, generating functional and skilled trained multi-state models using an automated process such as feature identification and extraction and subsequent training requires processing of millions of datapoints from input data and the generated features. This can require millions or billions of calculations to generate novel trained multi-state models from those millions of datapoints and millions or billions of calculations. As a result, each trained multi-state models is novel and distinct based on the input data and parameters of the algorithm. Thus, generating functional and skilled trained multi-state models comprises a process with a volume of calculation and analysis that a human brain cannot accomplish in a lifetime, or multiple lifetimes.

Similarly, the patient discharge disposition state system can be configured to continually receive data about the patients, perform the analysis, and provide periodic or continual updates via the disposition state report for each patient. This requires the analysis of thousands or millions of datapoints on a continual basis to optimize the reporting, requiring a volume of calculation and analysis that a human brain cannot accomplish in a lifetime.

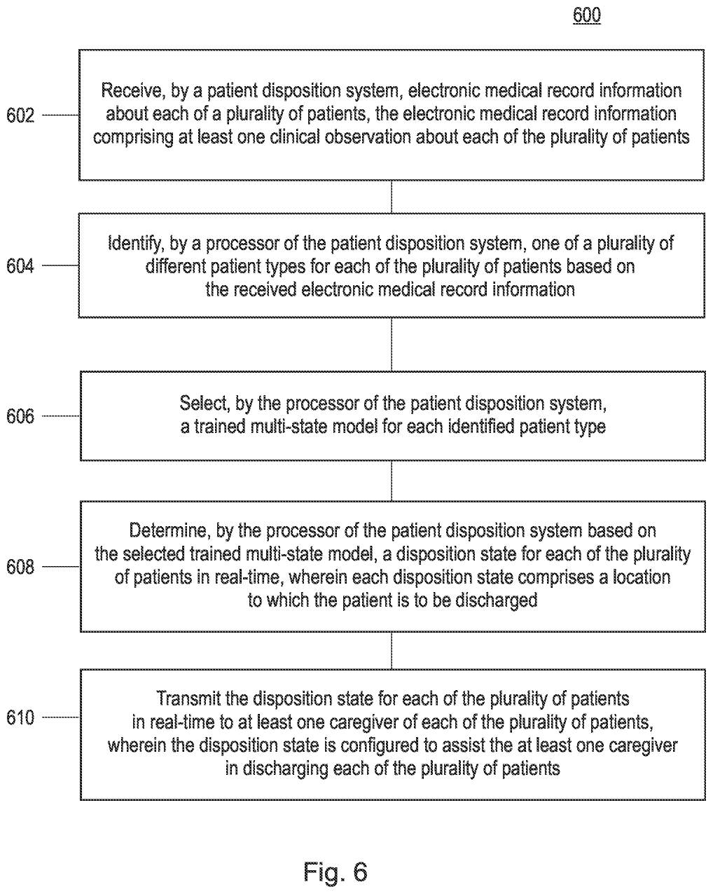

FIG. 6 is an example flowchart of a method for performing a patient disposition analysis for a plurality of patients using a patient disposition system. At step 602, a patient disposition system receives electronic medical record information about each of a plurality of patients. The electronic medical record information comprises at least one clinical observation about each of the plurality of patients.

At step 604, a processor of the patient disposition system identifies one of a plurality of different patient types (or clinical profiles) for each of the plurality of patients based on the received electronic medical record information.

At step 606, the processor of the patient disposition system selects a trained multi-state model for each identified patient type.

At step 608, the processor of the patient disposition system determines, based on the selected trained multi-state model, a disposition state for each of the plurality of patients in real-time. Wherein each disposition state comprises a location to which the patient is to be discharged.

At step 610, the patient disposition system transmits the disposition state for each of the plurality of patients in real-time to at least one caregiver of each of the plurality of patients. The disposition state is configured to assist the at least one caregiver in discharging each of the plurality of patients.

According to some embodiments, following step 610 at least one patient is discharged to the location.

According to some embodiments, the patient disposition system generates a patient disposition report comprising at least one determined disposition state for at least one patient of the plurality of patients in real-time. In embodiments, the patient disposition report is displayed in real-time on a display of the patient disposition system. The patient disposition report is also transmitted to a family member of the patient in real-time.

According to some embodiments, the patient disposition system receives or accesses sociodemographic data about the patient and information about post-hospital service providers. The processor of the patient disposition system determines at least one relevant or convenient service or assessment to be provided based on the information about the post-hospital service providers and the sociodemographic data about the patient. In embodiments, following the determination, medical services are obtained from at least one post-hospital service provider for the patient such that treatment is provided to the patient.

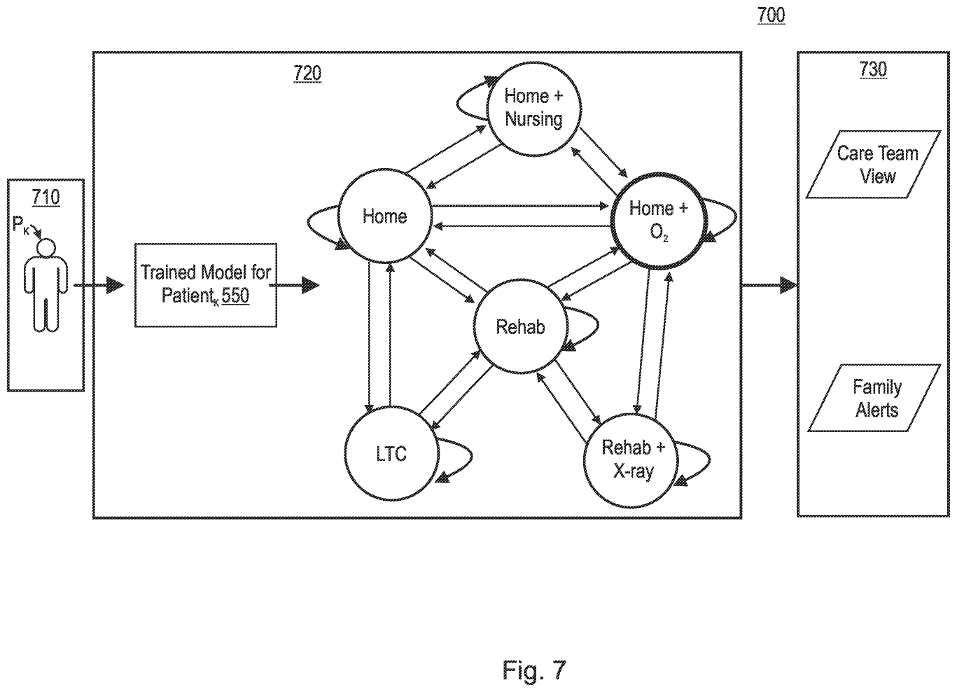

FIG. 7 is an example schematic representation of a patient disposition system 700. System 700 includes electronic medical record information 710 for a patient P_{K}, real-time multi-state prediction model 720, and means for user interaction 730. The medical record information 710 comprises the information described in step 120 in FIG. 1 and the information in step 210 in FIG. 2. The real-time prediction model 710 comprises any one of the multi-state models described herein for a particular patient type PATIENT_{K}. Means for user interaction 730 comprises the notifications described herein with reference to FIG. 4. The means for user interaction 730 can comprise the system sending a predicted disposition state of the patient in real-time to a caregiver of the patient (e.g., a care manager/care team). The means for user interaction 730 can also comprise the system sending the predicted disposition state of the patient in real-time to family members of the patient. The system shown in FIG. 7 helps the care team identify the most suitable discharge disposition state in real-time (e.g., discharge to home with home oxygen) for a given patient PATIENT_{K} and plan the discharge well in advance of the discharge. The system shown in FIG. 7 also helps the family of the patient to prepare for the discharge.

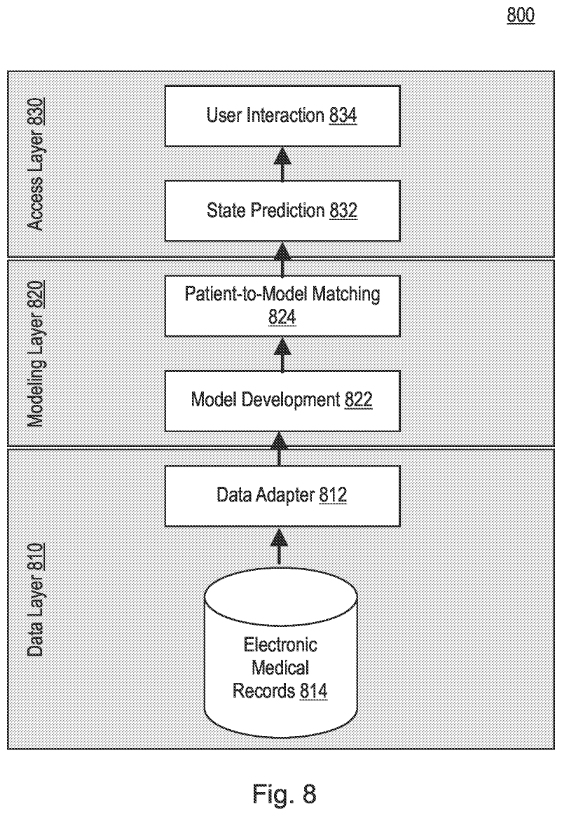

FIG. 8 is a schematic representation of a patient disposition system, in accordance with an embodiment. The system of FIG. 8 describes the system under three layers: a data layer 810, a modeling layer 820, and an access layer 830. Data layer 810 includes a data adapter module 812 that is configured to read all required electronic medical record data including clinical notes (e.g., sociodemographic, diagnosis, comorbidities, clinical events, insurance type, past hospital and ED visit data, etc.) from database 814 which is part of a hospital data repository/IT system. The data adapter module 812 puts the data into a common data format in real-time. In embodiments, the data is provided in a new input patient profile. This adapter module 812 also allows the system to easily interface the proposed prediction models with IT systems from different hospitals/facilities with minimal changes.

Modeling layer 820 comprises a model development module 822 that is configured to receive the data in common data format from the data adapter 812 and train multiple different multi-state models with the data. In embodiments, the models are Hidden Markov Models (HMI), a well-known statistical method for modeling stochastic dynamic systems. Model development module 822 trains the models based on the type of the patient as described in reference to FIG. 2. In embodiments, the model development module 822 uses only prior medical record data for targeted patient types in a particular hospital or institute for model building and validation. Patient-to-model matching module 824 of modeling layer 820 is configured to match the new input patient profile provided by the data layer 810 with the available trained models from the model development module 822. The matching module 824 reads all the required data elements based on the selected model on-the-fly and assists many different patient types in predicting discharge disposition states more accurately. In embodiments, patient-to-model matching module 824 is configured with a default model that applies when there is no match between one of the models and an identified patient type.

Access layer 830 includes a state prediction module 832 and a user interaction module 824. The state prediction module 832 is configured to provide a plug-and-play environment for the pre-trained multi-state prediction models from the model development module 822. A disposition state (i.e., disposition location: home, rehabilitation care, or long-term care along with the potential services/assessments that can be deferred) is generated by state prediction module 832 for a new patient in real-time using the corresponding multi-state model identified during the patient-to-model matching by module 824. The user interaction module 824 receives all of the predicted discharge disposition state information in real-time and generates different views based on the user. As referenced in FIGS. 4 and 7, the predicted disposition information is sent to a dashboard display for the case manager or care team including the potential services to be deferred. Additionally, based on the patient's demographics and economic factors, the user interaction module 834 helps the care team to match the best possible post-hospital care location, providers, and/or services. Plus, real-time phone and/or email alerts are sent to the patient's family members with the most up-to-date discharge disposition and post-hospital service requirements if any.

By providing earlier disposition state analyses in real-time, this novel patient discharge disposition system has an enormous positive effect on discharge planning compared to conventional systems and methods. As just one example, the system provides real-time dynamic predictions starting from the time of admission for a patient and the predictions improve in accuracy over time as new patient-specific data becomes available in the electronic medical record database. As another example, the system improves the communication between the patient's family and the care team with more timely predictions with anticipated discharge disposition. This helps to improve the discharge planning and related decision-making processes. The clinical care team and the patient's family, nursing home, or rehabilitation facility will be notified with potential services that can be deferred to the predicted discharge location. This helps to prepare for support after discharge and reduce the length of stay and cost of inpatient hospital stay. Additionally, the system advantageously provides a scalable framework that can assist many different patient types in predicting discharge disposition and post hospital services. The system provides plug-and-play type patient-specific predictive modules.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for performing, using a patient disposition system, a disposition analysis of a plurality of patients to optimize a discharge planning process for each of the plurality of patients, comprising:
receiving, by the patient disposition system, electronic medical record information about each of the plurality of patients, the electronic medical record information comprising at least one clinical observation about each of the plurality of patients;
identifying, by a processor of the patient disposition system, one of a plurality of different patient types for each of the plurality of patients based on the received electronic medical record information;
selecting, by the processor of the patient disposition system, a trained multi-state model for each identified patient type;
determining, by the processor of the patient disposition system based on the selected trained multi-state model, a disposition state for each of the plurality of patients in real-time, wherein each disposition state comprises a location to which the patient is to be discharged; and
transmitting the disposition state for each of the plurality of patients in real-time to at least one caregiver of each of the plurality of patients, wherein the disposition state is configured to assist the at least one caregiver in discharging each of the plurality of patients.

2. The method of claim 1, wherein a first patient of the plurality of patients is associated with at least one clinical observation that is indicative of a first patient type and the plurality of patients further comprises at least a second patient that is associated with at least one other clinical observation that is indicative of a second patient type, wherein the first and second patient types have different clinical profiles.

3. The method of claim 1, further comprising determining, by the processor of the patient disposition system, at least one service or assessment that can be deferred to the location to which at least one patient is to be discharged.

4. The method of claim 1, further comprising training at least one multi-state model for a first patient type, the training comprising: (i) identifying a first patient type based on electronic medical record information comprising patient information; (ii) extracting relevant data elements from the electronic medical record information; (iii) preprocessing the extracted relevant data elements to generate a clean dataset; (iv) splitting the clean dataset into a training data cohort (TRAIN) and a test data cohort (TEST); and (v) training the at least one multi-state model using the training data cohort.

5. The method of claim 4, wherein splitting the clean dataset into the training data cohort and the test data cohort comprises randomly splitting the clean dataset such that a first amount of the clean dataset in the training data cohort is different than a second amount of the clean dataset in the test data cohort.

6. The method of claim 4, wherein the training data cohort comprises a set of observation sequences and training the at least one multi-state model comprises determining at least one optimal parameter for maximizing a probability of observing the set of observation sequences.

7. The method of claim 1, further comprising generating, by the patient disposition system, a patient disposition report comprising at least one determined disposition state for at least one patient of the plurality of patients in real-time.

8. The method of claim 7, further comprising displaying the patient disposition report on a display of the patient disposition system in real-time or transmitting a notification regarding the at least one disposition state to a family member of the patient in real-time.

9. The method of claim 1, further comprising:
receiving, updated electronic medical record information for at least one patient of the plurality of patients;
updating, by the trained multi-state model, the disposition state for the at least one patient based on the received updated electronic medical record information; and
discharging the at least one patient to the location.

10. The method of claim 1, further comprising:
receiving or accessing, by the patient disposition system, sociodemographic data about the patient;
receiving or accessing, by the patient disposition system, information about post-hospital service providers;
determining, by the processor of the patient disposition system, at least one relevant or convenient service or assessment to be provided based on the information about the post-hospital service providers and the sociodemographic data about the patient; and
obtaining medical services from at least one post-hospital service provider for the patient such that treatment is provided to the patient.

11. A patient disposition system configured to perform a disposition analysis of a plurality of patients to optimize a discharge planning process for each of the plurality of patients, comprising:
a plurality of multi-state models, each trained to generate a disposition state for a patient;
electronic medical record information about the plurality of patients, the electronic medical record information comprising at least one clinical observation about each of the plurality of patients;
a processor configured to: (i) identify a patient type for each patient of the plurality of patients based on the electronic medical record information; (ii) select a trained multi-state model for each identified patient type; (iii) determine, based on the selected trained multi-state model, a disposition state for each patient of the plurality of patients in real-time, wherein each disposition state comprises a location to which the patient is to be discharged; and (iv) transmit the disposition state for each of the plurality of patients in real-time to at least one caregiver of each of the plurality of patients, wherein the disposition state is configured to assist the at least one caregiver in discharging each of the plurality of patients.

12. The patient disposition system of claim 11, wherein the plurality of patients comprises a first patient that is associated with at least one clinical observation that is indicative of a first patient type and the plurality of patients comprises at least a second patient that is associated with at least one other clinical observation that is indicative of a second patient type, wherein the first and second patient types have different clinical profiles.

13. The patient disposition system of claim 11, wherein the processor is further configured to determine at least one service or assessment that can be deferred to the location to which at least one patient is to be discharged.

14. The patient disposition system of claim 11, wherein the processor is further configured to generate a patient disposition report comprising at least one determined disposition state for at least one patient of the plurality of patients in real-time.

15. The patient disposition system of claim 14, further comprising a display configured to display the patient disposition report.
